(19) United States
(12) Patent Application Publication
(10) Pub. No.: US 2018/0030358 A1
(43) Pub. Date: Feb. 1, 2018

(54) PRODUCTION OF HIGHER HYDROCARBONS FROM SOLID BIOMASS

(71) Applicant: SHELL OIL COMPANY, Houston, TX (US)

(72) Inventors: Kimberly Ann JOHNSON, Richmond, TX (US); Ruth Mary KOWALESKI, Cypress, TX (US)

(21) Appl. No.: 15/659,980

(22) Filed: Jul. 26, 2017

Related U.S. Application Data
(60) Provisional application No. 62/367,761, filed on Jul. 28, 2016.

(51) Int. Cl. C10G 3/00; C07C 1/24; B01J 29/40

(52) U.S. Cl. CPC C10G 3/49; B01J 29/40; C07C 1/24; G01N 2030/025

(57) ABSTRACT

A process for the production of an aromatics-rich hydrocarbons useful as gasoline component from solid biomass is provided. The process provides for longer condensation catalyst life by contacting the stable oxygenated hydrocarbon intermediate produced from digestion and hydrodoxygenation of the solid biomass to a binder-free shaped ZSM-5 catalyst.

The present application claims the benefit of pending U.S. Provisonal Application No. 62/367,761, filed Jul. 28, 2016, the entire disclosure of which is hereby incorporation by reference.

### FIELD OF THE INVENTION

The invention relates to conversion of biomass to hydrocarbons. More specifically, the invention relates to improved production of higher hydrocarbons useful as liquid biofuels from solid biomass.

### BACKGROUND OF THE INVENTION

A significant amount of attention has been placed on developing new technologies for providing energy from resources other than fossil fuels. Biomass is a resource that shows promise as a fossil fuel alternative. As opposed to fossil fuel, biomass is also renewable.

Biomass may be useful as a source of renewable fuels. One type of biomass is plant biomass. Plant biomass is the most abundant source of carbohydrate in the world due to the lignocellulosic materials composing the cell walls in higher plants. Plant cell walls are divided into two sections, primary cell walls and secondary cell walls. The primary cell wall provides structure for expanding cells and is composed of three major polysaccharides (cellulose, pectin, and hemicellulose) and one group of glycoproteins. The secondary cell wall, which is produced after the cell has finished growing, also contains polysaccharides and is strengthened through polymeric lignin covalently cross-linked to hemicellulose. Hemicellulose and pectin are typically found in abundance, but cellulose is the predominant polysaccharide and the most abundant source of carbohydrates. However, production of fuel from cellulose poses a difficult technical problem. Some of the factors for this difficulty are the physical density of lignocelluloses (like wood) that can make penetration of the biomass structure of lignocelluloses with chemicals difficult and the chemical complexity of lignocelluloses that lead to difficulty in breaking down the long chain polymeric structure of cellulose into carbohydrates that can be used to produce fuel. Another factor for this difficulty is the nitrogen compounds and sulfur compounds contained in the biomass. The nitrogen and sulfur compounds contained in the biomass can poison catalysts used in subsequent processing.

Most transportation vehicles require high power density provided by internal combustion and/or propulsion engines. These engines require clean burning fuels which are generally in liquid form or, to a lesser extent, compressed gases. Liquid fuels are more portable due to their high energy density and their ability to be pumped, which makes handling easier.

Currently, bio-based feedstocks such as biomass provide the only renewable alternative for liquid transportation fuel. Unfortunately, the progress in developing new technologies for producing liquid biofuels has been slow in developing, especially for liquid fuel products that fit within the current infrastructure. Although a variety of fuels can be produced from biomass resources, such as ethanol, methanol, and vegetable oil, and gaseous fuels, such as hydrogen and methane, these fuels require either new distribution technologies and/or combustion technologies appropriate for their characteristics. The production of some of these fuels also tends to be expensive and raise questions with respect to their net carbon savings. There is a need to directly process biomass into liquid fuels, amenable to existing infrastructure.

Processing of biomass as feeds is challenged by the need to directly couple biomass hydrolysis to release sugars, and catalytic hydrogenation/hydrogenolysis/hydrodeoxygenation of the sugar, to prevent decomposition to heavy ends (caramel, or tars). Further, it is a challenge to minimize generation of waste products that may require treating before disposal and/or catalyst deactivation by poisons.

### SUMMARY OF THE INVENTION

It was found that oxygenated hydrocarbon intermediate produced by digesting and hydrodeoxygenating solid biomass in a liquid digestive solvent tend to rapidly coke the condensation catalyst in the subsequent condensation reaction that produces higher hydrocarbons. Applicants have found that contacting the oxygenated hydrocarbon intermediate with certain binder-free ZSM-5 catalysts as condensation catalysts provides longer catalyst life.

A process for the production of a higher hydrocarbon from solid biomass is provided, said process comprising:
a. providing a biomass solid containing cellulose, hemicellulose, and lignin;
b. digesting and hydrodeoxygenating the biomass solid in a liquid digestive solvent in the presence of a hydrothermal hydrocatalytic catalyst and hydrogen at a temperature in the range of 110° C. to less than 300° C. and at a pressure in a range of from 20 bar to 200 bar, to form a stable oxygenated hydrocarbon intermediate product, said stable oxygenated hydrocarbon intermediate product having at least 60% of carbon in molecules having 9 carbon atoms or less;
c. contacting at least a portion of the stable oxygenated hydrocarbon intermediate product with a binder-free shaped ZSM-5 catalyst having zeolite content of greater than 98%, silica to alumina molar ratio of at most 28 to 1, at a temperature in the range from 325° C. to about 425° C. producing water and an aromatics-rich higher hydrocarbons stream having at least 50 wt % of aromatics containing hydrocarbon based on the aromatics-rich hydrocarbons stream.

The features and advantages of the invention will be apparent to those skilled in the art. While numerous changes may be made by those skilled in the art, such changes are within the spirit of the invention.

### BRIEF DESCRIPTION OF THE DRAWING

The drawings illustrate certain aspects of some of the embodiments of the invention, and should not be used to limit or define the invention.

FIG. 1 is a schematic illustration of an embodiment of a process of this invention.

FIG. 2 is a plot of 2-methylbutene concentration (GC area %) over time (sample #) from Examples B, D and F and Comparative Example A.

FIG. 3 is a plot of xylenes concentration (GC area %) over time (sample #) from Examples B, D, E and F and Comparative Example A.

### DETAILED DESCRIPTION OF THE INVENTION

In one embodiment, the invention relates to contacting the oxygenated hydrocarbon intermediate, produced from digesting and hydrodeoxygenating a solid biomass in a liquid digestive solvent, with a binder-free shaped ZSM-5 catalyst having zeolite content of greater than 98%, silica to alumina molar ratio of at most 28 to 1, at a temperature in the range from 325° C. to about 425° C. producing water and an aromatics-rich higher hydrocarbons stream having at least 50 wt. % of aromatics containing hydrocarbon based on the aromatics-rich hydrocarbons stream.

The higher hydrocarbons produced are useful in forming transportation fuels, such as synthetic gasoline. As used herein, the term “higher hydrocarbons” refers to hydrocarbons having an oxygen to carbon ratio less than the oxygen to carbon ratio of at least one component of the biomass feedstock. The higher hydrocarbon predominantly contains C4 to C30 hydrocarbons, more preferably C6 to C18 hydrocarbons. As used herein the term “hydrocarbon” refers to an organic compound comprising primarily hydrogen and carbon atoms, which is also an unsubstituted hydrocarbon. In certain embodiments, the hydrocarbons of the invention also comprise heteroatoms (i.e., oxygen sulfur, phosphorus, or nitrogen) and thus the term “hydrocarbon” may also include substituted hydrocarbons. As used herein, the term “soluble carbohydrates” refers to monosaccharides or polysaccharides that become solubilized in a digestion process. Although the underlying chemistry is understood behind digesting cellulose and other complex carbohydrates and further transforming simple carbohydrates into organic compounds reminiscent of those present in fossil fuels, high-yield and energy-efficient processes suitable for converting cellulosic biomass into fuel blends have yet to be developed. In this regard, the most basic requirement associated with converting cellulosic biomass into fuel blends using digestion and other processes is that the energy input needed to bring about the conversion should not be greater than the available energy output of the product fuel blends. Further the process should maximize product yield while minimizing waste products. These basic requirements lead to a number of secondary issues that collectively present an immense engineering challenge that has not been solved heretofore.

Processing of biomass as feeds is challenged by the need to directly couple biomass hydrolysis to release sugars, and catalytic hydrogenation/hydrogenolysis/hydrodeoxygenation of the sugar, to prevent decomposition to heavy ends (caramel, or tars). It was found that some of the oxygenated hydrocarbon intermediate produced by digesting and catalytically hydrodeoxygenating solid biomass in a liquid digestive solvent tend to rapidly coke the condensation catalyst in the subsequent condensation reaction that produces higher hydrocarbons. It was found that contacting (and reacting) the oxygenated hydrocarbon intermediate with a certain binder-free shaped ZSM-5 having silica to alumina molar ratio of at most 28 to 1, have a longer catalyst life than a conventional ZSM-5 catalyst such as disclosed in US Patent Application Publication No. 2014/0173975.

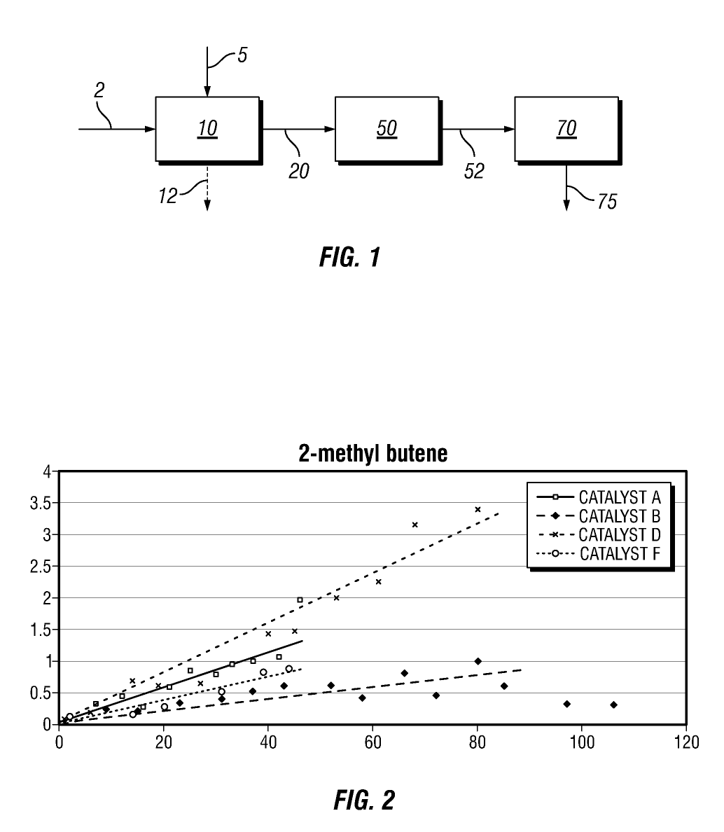

Various illustrative embodiments will be further described with reference to FIG. 1. In FIG. 1 show illustrative embodiments of biomass conversion process to hydrocarbon.

Any suitable (e.g., inexpensive and/or readily available) type of lignocellulosic biomass can be used as a solid biomass. Suitable lignocellulosic biomass can be, for example, selected from, but not limited to, wood, forestry residues, agricultural residues, herbaceous material, municipal solid wastes, pulp and paper mill residues, and combinations thereof. Thus, in some embodiments, the biomass can comprise, for example, corn stover, straw, bagasse, miscanthus, sorghum residue, switch grass, duckweed, bamboo, water hyacinth, hardwood, hardwood chips, hardwood pulp, softwood, softwood chips, softwood pulp, and/or combination of these feedstocks. The biomass can be chosen based upon a consideration such as, but not limited to, cellulose and/or hemicelluloses content, lignin content, growing time/season, growing location/transportation cost, growing costs, harvesting costs and the like.

Prior to processing, the untreated biomass can be reduced in size (e.g., chopping, crushing or debarking) to a convenient size and certain quality that aids in moving the biomass or mixing and impregnating the chemicals from digestive solvent. Thus, in some embodiments, providing biomass can comprise harvesting a lignocelluloses-containing plant such as, for example, a hardwood or softwood tree. The tree can be subjected to debarking, chopping to wood chips of desirable thickness, and washing to remove any residual soil, dirt and the like.

The biomass solids are introduced in to a vessel from an inlet. The vessel can be in any shape that include, for example, vertical, horizontal, incline, and may include bends, curves or u shape. The vessel will further have at least one inlet and at least one outlet.

The biomass may optionally be washed with an acidic or basic solution to remove metal species such as Mg, Ca, Na, K, Fe, Mn, and their corresponding anions such as chloride, sulfate, phosphate or nitrate that are detrimental to catalysts or equipment used in the hydrothermal hydrocatalytic treatment from the biomass. Such treatment disclosed in commonly owned co-pending US Patent application publication nos. US20150166681, US20150167236, US20150166682, US20150167241, US20150167238, US20150167235, US20150167240, US20150167237, US20150167239, and US20150184081, which disclosures are hereby incorporated by reference in its entirety.

At least a portion of the optionally treated cellulosic biomass solids is provided to a digestion and/or reaction zone (collectively referred to as “hydrothermal hydrocatalytic reaction zone”, for example represented as 10 in FIG. 1) for digesting and hydrodeoxygenating. This zone may be conducted in a single step or in multiple steps or vessels as described below.

For the hydrothermal catalytic reaction zone, the zone may have one or more vessels. In one embodiment, the digestion/reaction zone hydrolysis and hydrothermal hydrocatalytic reaction of the treated biomass is carried out in one or more vessels. These vessels may be digesters or reactors or combination thereof including a combination hydrothermal hydrocatalytic digestion unit.

In some embodiments, lignocellulosic biomass (solids), 2, being continuously or semi-continuously added to the hydrothermal digestion unit or hydrothermal hydrocatalytic digestion unit may be pressurized before being added to the unit, particularly when the hydrothermal (hydrocatalytic) digestion unit is in a pressurized state. Aqueous solution (or water) may be added with the biomass solids or separately. Pressurization of the cellulosic biomass solids from atmospheric pressure to a pressurized state may take place in one or more pressurization zones before addition of the cellulosic biomass solids to the hydrothermal (hydrocatalytic) digestion unit. Suitable pressurization zones that may be used for pressurizing and introducing lignocellulosic biomass to a pressurized hydrothermal digestion unit or hydrothermal hydrocatalytic digestion unit are described in more detail in commonly owned United States Patent Application Publications US20130152457 and US20130152458, and incorporated herein by reference in its entirety. Suitable pressurization zones described therein may include, for example, pressure vessels, pressurized screw feeders, and the like. In some embodiments, multiple pressurization zones may be connected in series to increase the pressure of the cellulosic biomass solids in a stepwise manner. The digestion and the hydrothermal hydrocatalytic reaction in the hydrothermal catalytic reaction zone (or digestion reaction zone) may be conducted separately, partially combined, or in situ.

The biomass solid is hydrothermally digested and hydrodeoxygenated in a liquid-phase digestive solvent, in the presence of hydrogen, for example via 5, and a catalyst capable of activating molecular hydrogen (hydrothermal hydrocatalytic catalyst) in a hydrothermal digestion unit, at a temperature in the range of from 110° C. to less than 300° C., and at a pressure in a range of from 20 bar to 200 bar to form stable oxygenated hydrocarbon intermediate product mixtures (plurality of oxygenated hydrocarbons). The stable oxygenated hydrocarbon intermediate product mixture, in general, has a viscosity of less than 100 centipoise (at 50° C.), a diol content, less than 2 wt. % of sugar, and less than 2 wt. % organic acid based on acetic acid equivalent, and at least 60% of carbon in formed product exists in molecules having 10 carbon atoms or less.

In some embodiments, the digestion rate of cellulosic biomass solids may be accelerated in the presence of a liquid phase containing a digestion solvent. In some instances, the liquid phase may be maintained at elevated pressures that keep the digestion solvent in a liquid state when raised above its normal boiling point. Although the more rapid digestion rate of cellulosic biomass solids under elevated temperature and pressure conditions may be desirable from a throughput standpoint, soluble carbohydrates may be susceptible to degradation at elevated temperatures. One approach for addressing the degradation of soluble carbohydrates during hydrothermal digestion is to conduct an in situ catalytic reduction reaction process so as to convert the soluble carbohydrates into more stable compounds as soon as possible after their formation.

In certain embodiments, a slurry catalyst may be effectively distributed from the bottom of a charge of cellulosic biomass solids to the top using upwardly directed fluid flow to fluidize and upwardly convey slurry catalyst particulates into the interstitial spaces within the charge for adequate catalyst distribution within the digesting cellulosic biomass solids. Suitable techniques for using fluid flow to distribute a slurry catalyst within cellulosic biomass solids in such a manner are described in commonly owned U. S. Patent Application Publication Nos. US20140005445 and US20140005444, which are incorporated herein by reference in its entirety. In addition to affecting distribution of the slurry catalyst, upwardly directed fluid flow may promote expansion of the cellulosic biomass solids and disfavor gravity-induced compaction that occurs during their addition and digestion, particularly as the digestion process proceeds and their structural integrity decreases. Methods of effectively distributing molecular hydrogen within cellulosic biomass solids during hydrothermal digestion is further described in commonly owned U. S. Patent Application Publication Nos. US20140174433 and US20140174432, which are incorporated herein by reference in its entirety.

In another embodiment the hydrothermal hydrocatalytic digestion unit may be configured as disclosed in a co-pending U. S. Application Publication No. US20140117276 which disclosure is hereby incorporated by reference. In the digestion zone, the size-reduced biomass is contacted with the digestive solvent where the digestion reaction takes place. The digestive solvent must be effective to digest lignins. The digestive solvent is typically a solvent mixture having a boiling point of at least 40° C.

In some embodiments, at least a portion of oxygenated hydrocarbons produced in the hydrothermal hydrocatalytic reaction zone are recycled within the process and system to at least, in part, form the in situ generated solvent, which is used in the biomass digestion process. Further, by controlling the rate of digestion of biomass to lower molecular weight fragments in the hydrothermal hydrocatalytic reaction (e.g., hydrogenolysis process), hydrogenation reactions can be conducted along with the hydrogenolysis reaction at temperatures ranging of from 110° C., preferably from about 150° C. to less than 300° C., most preferably from about 240° C. to about 270° C. As a result the fuel forming potential of the biomass feedstock fed to the process can be increased.

In various embodiments, the fluid phase digestion medium (liquid digestive solvent) in which the hydrothermal digestion and catalytic reduction reaction (in the hydrothermal hydrocatalytic reaction zone) are conducted, may comprise an organic solvent and water. The liquid digestive solvent mixture may have a normal boiling point (i.e., at atmospheric pressure) of at least 40° C., preferably at least 60° C., more preferably at least 80° C. Although any organic solvent that contains some oxygen atoms may be used as a digestion solvent, particularly advantageous organic solvents are those that can be directly converted into fuel blends and other materials and hence do not require extensive separation from intermediate streams used in the production of biofuels, or co-product streams used as fuel or separated and processed as chemical products. That is, particularly advantageous organic solvents are those that may be co-processed along with the alcoholic or oxygenated components during downstream processing reactions into fuel blends and other materials. Suitable organic solvents in this regard may include, for example, ethanol, ethylene glycol, propylene glycol, glycerol, phenolics and any combination thereof. In situ generated organic solvents are particularly desirable in this regard.

In some embodiments, the liquid phase digestive solvent may comprise between about 1% water and about 99% water. Although higher percentages of water may be more favorable from an environmental standpoint, higher quantities of organic solvent may more effectively promote hydrothermal digestion due to the organic solvent's greater propensity to solubilize carbohydrates and promote catalytic reduction of the soluble carbohydrates. In some embodiments, the liquid phase digestive solvent may comprise about 90% or less water by weight. In other embodiments, the fluid phase digestion medium may comprise about 80% or less water by weight, or about 70% or less water by weight, or about 60% or less water by weight, or about 50% or less water by weight, or about 40% or less water by weight, or about 30% or less water by weight, or about 20% or less water by weight, or about 10% or less water by weight, or about 5% or less water by weight.

In some embodiments, catalysts capable of activating molecular hydrogen hydrothermal hydrocatalytic catalysts, which are capable of activating molecular hydrogen (e.g., hydrogenolysis catalyst) and conducting a catalytic reduction reaction may comprise a metal such as, for example, Cr, Mo, W, Re, Mn, Cu, Cd, Fe, Co, Ni, Pt, Pd, Rh, Ru, Ir, Os, and alloys or any combination thereof, either alone or with promoters such as Au, Ag, Cr, Zn, Mn, Sn, Bi, B, O, and alloys or any combination thereof. In some embodiments, the catalysts and promoters may allow hydrogenation and hydrogenolysis reactions to occur at the same time or in succession of one another. In some embodiments, such catalysts may also comprise a carbonaceous pyropolymer catalyst containing transition metals (e.g., Cr, Mo, W, Re, Mn, Cu, and Cd) or Group VIII metals (e.g., Fe, Co, Ni, Pt, Pd, Rh, Ru, Ir, and Os). In some embodiments, the foregoing catalysts may be combined with an alkaline earth metal oxide or adhered to a catalytically active support. In some or other embodiments, the catalyst may be deposited on a catalyst support that may not itself be catalytically active.

In some embodiments, the hydrothermal hydrocatalytic catalyst may comprise a slurry catalyst. In some embodiments, the slurry catalyst may comprise a poison-tolerant catalyst. As used herein the term “poison-tolerant catalyst” refers to a catalyst that is capable of activating molecular hydrogen without needing to be regenerated or replaced due to low catalytic activity for at least about 12 hours of continuous operation. Use of a poison-tolerant catalyst may be particularly desirable when reacting soluble carbohydrates derived from cellulosic biomass solids that have not had catalyst poisons removed therefrom. Catalysts that are not poison tolerant may also be used to achieve a similar result, but they may need to be regenerated or replaced more frequently than does a poison-tolerant catalyst.

In some embodiments, suitable poison-tolerant catalysts may include, for example, sulfided catalysts. In some or other embodiments, nitrided catalysts may be used as poison-tolerant catalysts. Sulfided catalysts suitable for activating molecular hydrogen and buffers suitable for use with such catalysts are described in commonly owned U. S. Patent Application Publication Nos. US2012/0317872, US2013/0109896, US2012/0317873, and US20140166221, of which are incorporated herein by reference in its entirety. Sulfiding may take place by treating the catalyst with hydrogen sulfide or an alternative sulfiding agent, optionally while the catalyst is disposed on a solid support. In more particular embodiments, the poison-tolerant catalyst may comprise (a) sulfur and (b) Mo or W and (c) Co and/or Ni or mixtures thereof. The pH buffering agent, may be suitable be an inorganic salt, particularly alkali salts such as, for example, potassium hydroxide, sodium hydroxide, and potassium carbonate or ammonia. In other embodiments, catalysts containing Pt or Pd may also be effective poison-tolerant catalysts for use in the techniques described herein. When mediating in situ catalytic reduction reaction processes, sulfided catalysts may be particularly well suited to form reaction products comprising a substantial fraction of glycols (e.g., C2-C6 glycols) without producing excessive amounts of the corresponding monohydric alcohols. Although poison-tolerant catalysts, particularly sulfided catalysts, may be well suited for forming glycols from soluble carbohydrates, it is to be recognized that other types of catalysts, which may not necessarily be poison-tolerant, may also be used to achieve a like result in alternative embodiments. As will be recognized by one having ordinary skill in the art, various reaction parameters (e.g., temperature, pressure, catalyst composition, introduction of other components, and the like) may be modified to favor the formation of a desired reaction product. Given the benefit of the present disclosure, one having ordinary skill in the art will be able to alter various reaction parameters to change the product distribution obtained from a particular catalyst and set of reactants.

In some embodiments, slurry catalysts suitable for use in the methods described herein may be sulfided by dispersing a slurry catalyst in a fluid phase and adding a sulfiding agent thereto. Suitable sulfiding agents may include, for example, organic sulfoxides (e.g., dimethyl sulfoxide), hydrogen sulfide, salts of hydrogen sulfide (e.g., NaSH), and the like. In some embodiments, the slurry catalyst may be concentrated in the fluid phase after sulfiding, and the concentrated slurry may then be distributed in the cellulosic biomass solids using fluid flow. Illustrative techniques for catalyst sulfiding that may be used in conjunction with the methods described herein are described in United States Patent Application Publication US2010/0236988 and incorporated herein by reference in its entirety.

In various embodiments, slurry catalysts used in conjunction with the methods described herein may have a particulate size of about 250 microns or less. In some embodiments, the slurry catalyst may have a particulate size of about 100 microns or less, or about 10 microns or less. In some embodiments, the minimum particulate size of the slurry catalyst may be about 1 micron. In some embodiments, the slurry catalyst may comprise catalyst fines in the processes described herein.

Catalysts that are not particularly poison-tolerant may also be used in conjunction with the techniques described herein. Such catalysts may include, for example, Ru, Pt, Pd, or compounds thereof disposed on a solid support such as, for example, Ru on titanium dioxide or Ru on carbon. Although such catalysts may not have particular poison tolerance, they may be regenerable, such as through exposure of the catalyst to water at elevated temperatures, which may be in either a subcritical state or a supercritical state.

In some embodiments, the catalysts used in conjunction with the processes described herein may be operable to generate molecular hydrogen. For example, in some embodiments, catalysts suitable for aqueous phase reforming (i.e., APR catalysts) may be used. Suitable APR catalysts may include, for example, catalysts comprising Pt, Pd, Ru, Ni, Co, or other Group VIII metals alloyed or modified with Re, Mo, Sn, or other metals such as described in United States Patent Publication US2008/0300435 and incorporated herein by reference in its entirety.

As described above, one or more liquid phases may be present when digesting cellulosic biomass solids. Particularly when cellulosic biomass solids are fed continuously or semi-continuously to the hydrothermal (hydrocatalytic) digestion unit, digestion of the cellulosic biomass solids may produce multiple liquid phases in the hydrothermal digestion unit. The liquid phases may be immiscible with one another, or they may be at least partially miscible with one another. In some embodiments, the one or more liquid phases may comprise a phenolics liquid phase comprising lignin or a product formed therefrom, an aqueous phase comprising the alcoholic component, a light organics phase, or any combination thereof. The alcoholic component being produced from the cellulosic biomass solids may be partitioned between the one or more liquid phases, or the alcoholic component may be located substantially in a single liquid phase. For example, the alcoholic component being produced from the cellulosic biomass solids may be located predominantly in an aqueous phase (e.g., an aqueous phase digestion solvent), although minor amounts of the alcoholic component may be partitioned to the phenolics liquid phase or a light organics phase. In various embodiments, the slurry catalyst may accumulate in the phenolics liquid phase as it forms, thereby complicating the return of the slurry catalyst to the cellulosic biomass solids in the manner described above. Alternative configurations for distributing slurry catalyst particulates in the cellulosic biomass solids when excessive catalyst accumulation in the phenolics liquid phase has occurred are described hereinafter.

Accumulation of the slurry catalyst in the phenolics liquid phase may, in some embodiments, be addressed by conveying this phase and the accumulated slurry catalyst therein to the same location where a fluid phase digestion medium is being contacted with cellulosic biomass solids. The fluid phase digestion medium and the phenolics liquid phase may be conveyed to the cellulosic biomass solids together or separately. Thusly, either the fluid phase digestion medium and/or the phenolics liquid phase may motively return the slurry catalyst back to the cellulosic biomass solids such that continued stabilization of soluble carbohydrates may take place. In some embodiments, at least a portion of the lignin in the phenolics liquid phase may be depolymerized before or while conveying the phenolics liquid phase for redistribution of the slurry catalyst. At least partial depolymerization of the lignin in the phenolics liquid phase may reduce the viscosity of this phase and make it easier to convey. Lignin depolymerization may take place chemically by hydrolyzing the lignin (e.g., with a base) or thermally by heating the lignin to a temperature of at least about 250° C. in the presence of molecular hydrogen and the slurry catalyst. Further details regarding lignin depolymerization and the use of viscosity monitoring as a means of process control are described in commonly owned U. S. Patent Application Publication No. US20140117275 which disclosure is incorporated herein by reference in its entirety.

In some embodiments, a phenolics liquid phase formed from the cellulosic biomass solids may be further processed. Processing of the phenolics liquid phase may facilitate the catalytic reduction reaction being performed to stabilize soluble carbohydrates. In addition, further processing of the phenolics liquid phase may be coupled with the production of glycols or dried monohydric alcohols for feeding to a condensation catalyst. Moreover, further processing of the phenolics liquid phase may produce methanol and phenolic compounds from degradation of the lignin present in the cellulosic biomass solids, thereby increasing the overall weight percentage of the cellulosic biomass solids that may be transformed into useful materials. Finally, further processing of the phenolics liquid phase may improve the lifetime of the slurry catalyst.

Various techniques for processing a phenolics liquid phase produced from cellulosic biomass solids are described in commonly owned U. S. Patent Application Publication Nos. US20140121419, US20140117277, which disclosures are incorporated herein by reference in its entirety. As described therein, in some embodiments, the viscosity of the phenolics liquid phase may be reduced in order to facilitate conveyance or handling of the phenolics liquid phase. As further described therein, deviscosification of the phenolics liquid phase may take place by chemically hydrolyzing the lignin and/or heating the phenolics liquid phase in the presence of molecular hydrogen (i.e., hydrotreating) to depolymerize at least a portion of the lignin present therein in the presence of accumulated slurry catalyst. Deviscosification of the phenolics liquid phase may take place before or after separation of the phenolics liquid phase from one or more of the other liquid phases present, and thermal deviscosification may be coupled to the reaction or series of reactions used to produce the alcoholic component from the cellulosic biomass solids. Moreover, after deviscosification of the phenolics liquid phase, the slurry catalyst may be removed therefrom. The catalyst may then be regenerated, returned to the cellulosic biomass solids, or any combination thereof. In some embodiments, heating of the cellulosic biomass solids and the fluid phase digestion medium (liquid digestive solvent) to form soluble carbohydrates and a phenolics liquid phase may take place while the cellulosic biomass solids are in a pressurized state. As used herein, the term “pressurized state” refers to a pressure that is greater than atmospheric pressure (1 bar). Heating a fluid phase digestion medium in a pressurized state may allow the normal boiling point of the digestion solvent to be exceeded, thereby allowing the rate of hydrothermal digestion to be increased relative to lower temperature digestion processes. In some embodiments, heating the cellulosic biomass solids and the fluid phase digestion medium may take place at a pressure of at least about 30 bar. In some embodiments, heating the cellulosic biomass solids and the fluid phase digestion medium may take place at a pressure of at least about 60 bar, or at a pressure of at least about 90 bar. In some embodiments, heating the cellulosic biomass solids and the fluid phase digestion medium may take place at a pressure ranging between about 30 bar and about 430 bar. In some embodiments, heating the cellulosic biomass solids and the fluid phase digestion medium may take place at a pressure ranging between about 50 bar and about 330 bar, or at a pressure ranging between about 70 bar and about 130 bar, or at a pressure ranging between about 30 bar and about 130 bar.

As one example of digestion and hydrodeoxygenation of lignocellulosic biomass, the following procedure was used. A 50-milliliter Parr 4590 reactor was charged with 6.01 grams of tetrahydrofuran and 17.99 grams of deionized water solvent, together with 0.099 grams of potassium hydroxide, and 0.1075 grams of Raney™ cobalt catalyst (from WR Grace 2724).

The reactor was then charged with 1.99 grams of southern pine mini-chips (10% moisture), of nominal size 3×5×5 mm in dimension, before pressuring with 52 bar of hydrogen, and heating with stirring to 190° C. for 1 hour, followed by heating to 240° C. for 4 hours. At the end of the 5-hour reaction cycle, the reactor was cooled, and allowed to gravity settle overnight.

The reaction cycle was repeated three times via addition of 2 more grams of wood chips, and re-pressuring with 52 bar of H₂ before heating using the same temperature profile.

After four cycles, the reactor product was analyzed by gas chromatography using a 60-m×0.32 mm ID DB-5 column of 1 micrometer thickness, with 50:1 split ratio, 2 ml/min helium flow, and column oven at 40° C. for 8 minutes, followed by ramp to 285° C. at 10° C./min, and a hold time of 53.5 minutes. The injector temperature was set at 250° C., and the detector temperature was set at 300° C. A range of alkanes, ketone and aldehyde monooxygenates as well as glycol intermediates including ethylene glycol (EG), 1,2-propylene glycol (PG) and glycerol were observed. Total products observed in the gas chromatographic analysis summed to about 30% of the maximum expected yield if all carbohydrates were converted to mono-oxygenated or diol products. Ethylene glycol (EG) formation and 1,2-propylene glycol (PG) formation comprised approximately 20% of observed products. All observed reaction products exhibited volatility greater than C6 sugar alcohol sorbitol.

The digestion and hydrodeoxygenation of the biomass solid described herein produces a stable oxygenated hydrocarbon intermediate product (for example 20 in FIG. 1), that has a viscosity of less than 100 centipoise (at 50° C.), preferably less than 40 centipoise, containing a plurality of oxygenated hydrocarbons (may contain diol, and preferably less than 2 wt % of sugar, and less than 2 wt % acid based on acetic acid equivalent, based on the total stream composition), and at 60% of carbon exists in molecules having 9 carbon atoms or less. By the term “stable”, the product is stable enough to be stored for at least 30 days where the viscosity does not change more than 50% and the main components (top 10 percent based on mass basis) do not change in concentration by more than 10%.

Optionally, the stable oxygenated hydrocarbon intermediate product (plurality of oxygenated hydrocarbons) can be vaporized to allow ash separation from the liquid product. (Optional separation of ash for example as 12 in FIG. 1). The vaporized stable oxygenated hydrocarbon can then be provided to the further conversion zone which includes condensation (for example 50 in FIG. 1) described below.

Optionally in such conversion zone, at least a portion of the stable oxygenated hydrocarbon intermediate product may be contacted, in a diol conversion zone, with an acidic amorphous silica alumina catalyst at a temperature in the range from 300° C. to 400° C., preferably 325° C. to 375° C., thereby producing a monooxygentaed stream, as described in commonly owned U.S. Patent Applications 62/186,941, 62/186902, 62/186919, 62/186960, all filed on Jun. 30, 2015, each of which is incorporated herein by reference in its entirety. The temperature and pressure is at a range that optimizes diol conversion while minimizing coke formation (by oligomerization or condensation reactions). The pressure range may be from ambient pressure (atmospheric) to slight partial pressure, for example, total pressure of up to about 200 psi. The reaction typically converts at least 25%, preferably at least 50%, most preferably at least 75% of diols initially present. Typically, the weight hourly space velocity is in the range of 0.2 to 5 for the monooxygenate formation step. Solid acid amorphous silica-alumina catalyst is available commercially, for example, from Criterion Catalyst Co., such as X-600 catalyst series, X-503 catalyst, X-801 catalyst or from CRI Catalyst Company such as KL-7122 catalyst.

The monooxygenated stream can be optionally be condensed (in this instance referred to liquid condensation without chemical transformation) in a cooling zone, to liquid producing an aqueous phase and an organic phase. The monooxygenated stream optionally can be phase separated into an aqueous phase and an organic phase upon condensation, thus allowing the aqueous phase containing water and a residual amount of unconverted monooxygenated compounds and diols of carbon number less than four, to be readily removed from the organic phase enriched in monooxygenated organic compounds greater than carbon number four, and phenolic compounds. Optionally, at least a (first) portion of the organic phase can optionally be recycled to the hydrothermal catalytic reaction zone (digestion and hydrodeoxygenation) as a portion of the digestive solvent.

As used herein, the term “condensation reaction” will refer to a chemical transformation in which two or more molecules are coupled with one another to form a carbon-carbon bond in a higher molecular weight compound, usually accompanied by the loss of a small molecule such as water or an alcohol. The term “condensation catalyst” will refer to a catalyst that facilitates, causes or accelerates such chemical transformation.

In the inventive process, at least a (second) portion of the organic phase containing the monooxygenates or the monooxygenated stream is contacted with a binder-free shaped ZSM-5 catalyst having zeolite content of greater than 98%, silica to alumina molar ratio of at most 28 to 1, at a temperature in the range from 325° C. to about 425° C. preferably 350° C. to 400° C., in the condensation reaction zone, 50, producing water and an aromatics-rich higher hydrocarbons stream having at least 50 wt % of aromatics containing hydrocarbon based on the aromatics-rich hydrocarbons stream. The entire organic phase can also be sent to the condensation step. The temperature and pressure is at a range that optimizes condensation reaction while minimizing coke formation. The pressure range may be from ambient pressure (atmospheric) to slight partial pressure, for example, total pressure of up to about 200 psi. The yield may be greater than 40% of carbons based on biomass carbons due to the increase catalyst uptime (amount of monooxygenated stream passed over the condensation catalyst). Aromatics as defined herein can be quantified by GC-MS analysis and includes any aromatic containing hydrocarbon that contains aromatic rings that are not oxygenated, such as mesytilene, based on molecular content.

During the course of the condensation reaction, coke formation occurs. For biomass-derived materials as feed, such as in the inventive process, the rate of coke formation is higher than convention petroleum hydrocarbons. The presence of coke on the catalyst causes temporary loss of activity. This temporary deactivation is reversed by oxidative regeneration or coke burn. During the coke burn, typically the normal feed stream of monooxygenates is stopped while a mixture of nitrogen and oxygen is passed over the catalyst at high temperature (e.g., >400° C.). Precise conditions for this step vary somewhat and are optimized for given process and type of coke. In general, the oxidative regeneration may be carried out at a temperature effective to burn the coke in the presence of oxygen containing gas (e.g., air, mixtures of oxygen and inert gas such as nitrogen or argon). The regeneration temperature may be about equal or greater than 390° C., preferably about equal or greater than 400° C. The temperature may be much higher as long as economically feasible as long as the integrity of the ZSM-5 catalyst is maintained, up to about 700° C. General regeneration of ZSM-5 may be found in U.S. Pat. No. 5,648,585, U.S. Pat. No. 8,916,490, or U.S. Pat. No. 8,946,106 which disclosure is hereby incorporated by reference. Pressure may be atmospheric pressure or above. For example, in the examples described in the illustrative examples below, a mixture of 5% oxygen/95% nitrogen at 100 psig flowed through the catalyst at 100 cc/min for 48 hrs at 400° C. At the conclusion of the 48 hrs, the reactor was returned to the normal process temperature, gas flow was switched from oxygen/nitrogen to pure nitrogen at 50 cc/min and pressure was reduced to 200 psig. Normal feed forward was resumed.

Over the lifetime of a catalyst, the coke burns become progressively less effective as a portion of active sites are lost to permanent deactivation. While not wishing to be bound by theory, the cause of this permanent loss of activity is thought be exposure to steam in the process (hydrothermal degradation) as discussed for example in “Processing Bomass-Derived Oxygenates in the Oil Refinery: Catalytic Cracking (FCC) Reaction Pathways and Role of Catalyst”, by Avelino Corma, George W. Huber, Laurent Sauvanaud, P.O. Conner, Journal of Catalysis, Vol. 247 (2007) pages 307-327. The figures and table below show this permanent loss in activity as an increase in olefin, decrease in aromatics (xylene) formation, and a reduction in fuel yield.

The advantage of using the novel binder-free ZSM-5 in the process of the invention is that it has shown higher hydrothermal stability, (i.e., resistance to the permanent deactivation). The novel catalyst continued to produce aromatic fuel much longer (i.e., longer catalyst life) than the conventional catalyst, an alumina bound ZSM-5. It has been shown that the binder-free ZSM-5 catalyst used in the condensation process of a biomass-derived oxygenates may preferably be regenerated at least 10 times, more preferably at least 12 times, more preferably at least 15 times, more preferably at least 20 times.

The binder-free shaped ZSM-5 catalyst useful for the invention has zeolite content of greater than 98%, and is made without addition of binders (binder-less 100% zeolite catalyst). The binder-free shaped ZSM-5 catalyst is preferably a binder-free ZSM-5 extrudate. The silica to alumina molar ratio of the of binder-free shaped ZSM-5 catalyst at most 28 to 1, preferably of at most 27 to 1, more preferably at most 26:1, even more preferably at most 25:1. The binder-free shaped ZSM-5 catalyst preferably has a sodium oxide content (Na₂O) of less than 0.15 wt. %, more preferably at most 0.10 wt. %. (Sodium oxide content is determined assuming all sodium present in the completely digested sample is present as sodium oxide) In some instances, the binder-free shaped ZSM-5 catalyst may preferably have a mesopore volume for pore widths in the range of 50 Å to 1000 Å of at least 0.070 cc/g. The binder-free shaped ZSM-5 catalyst preferably has a BET surface area in the range of 300 m²/g to 500 m²/g, more preferably in the range of 350 m²/g to 450 m²/g. The catalyst typically has a crush strength of 0.5 lb/mm or greater, preferably 1 lb/mm or greater. The binder-free shaped ZSM-5 catalyst preferably has a Brønsted acidity of at least 0.80 mmole/g, more preferably acidity of at least 0.85 mmole/g.

The binder-free shaped ZSM-5 catalyst may be prepared as described in U.S. Pat. No. 5,558,851, U.S. Pat. No. 6,261,534 and U.S. Pat. No. 6,632,415, which disclosures are hereby incorporated by reference. The binder-free shaped ZSM-5 catalyst may be prepared with or without use of a template in the reaction mixture. These methods allow preparation of a ZSM-5 extrudate that contains 100% ZSM-5 without the use of a binder. The binder-free shaped ZSM-5 is defined by the presence of the X-ray diffraction lines in Table 1.

For example, a crystalline zeolite extrudate having the X-ray diffraction lines of Table 1 can be prepared from a mixture containing at least one active source of silicon oxide and a second oxide of aluminum. The mixture is heated for a time sufficient to form a crystalline zeolite with the X-ray diffraction lines of Table 1 wherein the molar ratio of SiO₂/Al₂O₃ in the mixture ranges from 20-50. The heating occurs in the absence of an external liquid phase where the molar ratio of H₂O/SiO₂ is less than about 8. The amount of water used is less than the amount of water required for conventional zeolite preparation processes. After the crystallization, there is no separate liquid phase which must be removed prior to washing the crystalline solid. The amount of water used should also be low enough to prevent the shaped extrudates from collapsing or “melting” during the crystallization.

Possible sources of aluminum oxide in the reaction mixture includes aluminates, aluminas such as AlOOH or Al(OH)₃, other zeolites, clays, or aluminum compounds such as Al₂(SO₄)₃. Sodium aluminate and aluminum hydroxide are preferred.

Possible sources of silicon oxide include precipitated silica, silicates, silica hydrogel, silicic acid, colloidal silica, fumed silica, tetralkylorthosilicates, hydroxides, aluminosilicates and silica-aluminas.

The amount of water used in the in-extrudate crystallization is significantly less than that used in conventional synthesis. The initial H₂O/SiO₂ in the reaction mixture is preferably less than about 8. The water and other liquid ingredients (such as solutions of, NaOH and optionally TPABr or TPAOH) added should be sufficient to allow wetting of all of the dry ingredients and formation of a uniform paste upon mixing with heat. The mixture may be heated further to dry the mixture to a consistency that will allow extrusion.

In addition to silica, alumina and water, the reaction mixture may contain one or more sources of alkali metal oxide such as alkali oxides, hydroxides, nitrates, sulfates, halogenides, oxalates, citrates or acetates. The molar ratio of alkali (M⁺) to silica may range from 0 to 1.5 and is preferably between 0.1 and 0.3. The alkali metal compound may also contribute OH⁻. The OH⁻/SiO₂ molar ratio includes OH— from all sources including metal and organic hydroxides and aluminum compounds which release OH— is generally 0.1-0.4 and preferably between 0.1 and 0.3.

Zeolite synthesis often occurs in the presence of organic templates which are known in the art as structure directing agents (SDA), although some zeolites can be synthesized without SDA. For ZSM-5 synthesis, a typical SDA used is tetrapropylammonium bromide (TPABr) or tetrapropylammonium hydroxide (TPAOH). The TPA/SiO₂ molar ratio may range from 0 to 0.9 and is most typically 0.0 to 0.3.

Seed crystals containing the desired zeolite may be added to the reaction mixture to facilitate formation of the zeolite although they are not required. When used, seed crystals are added at a level of about 2-10% based on the weight of the dry ingredients.

An advantage of the in-extrudate synthesis is that the raw materials may be formed into the desired shape prior to crystallization. If necessary, additional water may be added to form an extrudable mix. The optimum extrusion moisture will depend on the raw materials used in the mixture. The LOI (loss on ignition at 550° C.) of the extrusion mix may range from about 48-65%. The cross sectional diameter of the extrudates is preferentially between 1/32″ and ¼″ diameter.

After extrusion the extrudate may be crystallized as is or dried further to allow for the optimum crystallization. The optimum LOI prior to crystallization may range from 30-65% but is preferably about 35-50% for ZSM-5. No additional water beyond what is required to form the extrudate is needed for crystallization.

The crystallization takes place in the absence of an external liquid phase at elevated temperature in an autoclave at autogeneous pressure. The temperatures during the hydrothermal crystallization step are maintained from about 100 to 180° C. and preferably between 140 and 170° C. for ZSM-5 synthesis. Due to the low levels of moisture, crystallization is typically accelerated relative to conventional synthesis methods using lower solids content. The crystallization time require may range from 3 hours to 10 days and is preferably between 1 and 3 days. The resulting solids will typically comprise at least 80% zeolite and more frequently 95-100% zeolite. The hydrothermal crystallization may occur in a closed vessel, such as an autoclave, so that the crystallization occurs at autogenous pressure.

The crystallized zeolite is washed with neutral, acidic or basic solution (dilute HNO₃ or NaOH) to remove amorphous material, especially SiO₂, from the zeolite. After washing, the zeolite is dried at 90-120° C. for 8-24 hours in air.

Ion-exchange is used to replace sodium in the zeolite with hydrogen, ammonium, or others ions. Typically, the ion exchange will involve contacting the zeolite with a solution of ammonium hydroxide, ammonium chloride, or ammonium sulfate. Multiple contacts may be used. The zeolite may be calcined before or after ion exchange. Calcination may be done at temperatures ranging from 200-800° C. The ammonium form of the zeolite is converted to the hydrogen form by calcination.

The condensation reaction mediated by the condensation catalyst may be carried out in any reactor of suitable design, including continuous-flow, batch, semi-batch or multi-system reactors, without limitation as to design, size, geometry, flow rates, and the like. The reactor system may also use a fluidized catalytic bed system, a swing bed system, fixed bed system, a moving bed system, or a combination of the above. In some embodiments, bi-phasic (e.g., liquid-liquid) and tri-phasic (e.g., liquid-liquid-solid) reactors may be used to carry out the condensation reaction. In various embodiments, the higher molecular weight compound produced by the condensation reaction (higher hydrocarbons) may comprise >C4 hydrocarbons. In some or other embodiments, the higher molecular weight compound produced by the condensation reaction may comprise >C6 hydrocarbons. In some embodiments, the higher molecular weight compound produced by the condensation reaction may comprise C4-C30 hydrocarbons. In some embodiments, the higher molecular weight compound produced by the condensation reaction may comprise C6-C30 hydrocarbons. In still other embodiments, the higher molecular weight compound produced by the condensation reaction may comprise C4-C24 hydrocarbons, or C6-C24 hydrocarbons, or C4-C18 hydrocarbons, or C6-C18 hydrocarbons, or C4-C12 hydrocarbons, or C6-C12 hydrocarbons. As used herein, the term “hydrocarbons” refers to compounds containing both carbon and hydrogen without reference to other elements that may be present. Thus, heteroatom-substituted compounds are also described herein by the term “hydrocarbons.” Under the present inventive process, the higher hydrocarbons contains aromatic compounds.

The aromatics-rich higher hydrocarbon stream, 52, can optionally be washed with aqueous base such as sodium hydroxide, potassium hydroxide to remove residual acids and phenolics (washing zone, 70) to produce biofuel useful as gasoline, 75. These aqueous base typically have a pH of at least 9, preferably at least 10.

In one embodiment, a process for the production of a higher hydrocarbon from solid biomass is provided comprising:
a. providing a biomass solid containing cellulose, hemicellulose, and lignin;
b. digesting and hydrodeoxygenating the biomass solid in a liquid digestive solvent in the presence of a hydrothermal hydrocatalytic catalyst and hydrogen at a temperature in the range of 110° C. to less than 300° C. and at a pressure in a range of from 20 bar to 200 bar, to form a stable oxygenated hydrocarbon intermediate product, said stable oxygenated hydrocarbon intermediate product having at least 60% of carbon in molecules having 9 carbon atoms or less;
c. contacting at least a portion of the stable oxygenated hydrocarbon intermediate product with a binder-free shaped ZSM-5 catalyst having zeolite content of greater than 98%, silica to alumina molar ratio of at most 28 to 1 and an acidity of at least 0.80 mmole/g., at a temperature in the range from 325° C. to about 425° C. producing water and an aromatics-rich higher hydrocarbons stream having at least 50 wt % of aromatics containing hydrocarbon based on the aromatics-rich hydrocarbons stream;
d. regenerating the binder-free shaped ZSM-5 catalyst from step (c) at a temperature of about equal or greater than 390° C. in the presence of an oxygen-containing gas thereby producing a regenerated catalyst and further conducting step (c) using such regenerated catalyst. All the features and measures described above and/or below are functionally independent and may be equally or similarly applied independently to the embodiment.

While the invention is susceptible to various modifications and alternative forms, specific embodiments thereof are shown by way of examples herein described in detail. It should be understood, that the detailed description is not intended to limit the invention to the particular form disclosed, but on the contrary, the intention is to cover all modifications, equivalents and alternatives falling within the spirit and scope of the present invention as defined by the appended claims. The person skilled in the art will readily understand that, while the invention is illustrated making reference to one or more a specific combinations of features and measures, many of those features and measures are functionally independent from other features and measures such that they can be equally or similarly applied independently in other embodiments or combinations.

To facilitate a better understanding of the present invention, the following examples of preferred embodiments are given. In no way should the following examples be read to limit, or to define, the scope of the invention.

### ILLUSTRATIVE EXAMPLES

### Test Methods

### Crush Strength:

Crush strength is measured by averaging the flat plate crush strength of 40 dried extrudate pellets and dividing by the average extrudate length. Crush strength is measured using a Dillon Quantrol™ TC2 i-series Computer Controlled Force Test Stand and average length is determined using the Advanced Laboratory Imaging Analysis System.

### XRD

The XRD analysis of ZSM-5 samples was performed using a PANalytical X'pert Pro MPD, powered by Empyrean Cu LFF HR DK386813 X-ray tube and fitted with a PIXcellD line detector. XRD sample preparation involved gentle grinding of ZSM-5 samples into fine powder and back-packing of approximately 0.5 g of the sample into an XRD sample holder with light compression to make it flat and tight. Samples were exposed to Ni-filtered Cu Kα-rays radiation source operated at 45 kV and 40 mA. The powder X-ray diffraction patterns of the samples were collected over 20 range of 5° to 50° at a step size of 0.0131° and an effective scanning speed of 61.20 sec per step, and they were measured in Bragg-Brentano reflection geometry. During XRD scans, soller slits of 0.04 rad, fixed anti-scatter of 2°, and programmable divergence slits of 10 mm irradiated length were used together with a fixed incident beam mask of 10 m. Their phase identification was carried out by means of the PANalytical X'Pert accompanying software program, PANalytical HighScore Plus, in conjunction with the International Centre for Diffraction Data (ICDD) Powder Diffraction File (PDF-4+, edition 2015) and the Crystallography Open Database (2015; www.crystallography.net).

The X-ray diffraction peaks and relative intensities are shown in the table below and are characteristic of ZSM-5 zeolite. In the table below, VS=very strong, S=strong, M=medium and W=weak intensity.

TABLE 1

| d-spacing [Å] | Relative Intensity (I/Io) |
|---|---|
| 11.16 | VS |
| 9.97 | S |
| 9.75 | W |
| 6.72 | W |
| 6.38 | W |
| 6.00 | W |
| 5.72 | W |
| 5.59 | W |
| 5.04 | W |
| 4.99 | W |
| 4.63 | W |
| 4.38 | W |
| 4.28 | W |
| 4.27 | W |
| 4.02 | W |
| 3.86 | VS |
| 3.83 | VS |
| 3.76 | M |
| 3.73 | M |
| 3.66 | M |
| 3.49 | W |
| 3.46 | W |
| 3.44 | W |
| 3.38 | W |
| 3.36 | W |
| 3.32 | W |
| 3.06 | W |
| 3.00 | W |

### Surface Area and Porosity Measurements

N2 adsorption at 77 K was carried out in a Micromeritics ASAP 2420 apparatus. Samples were calcined at 500° C. for one hour and evacuated at 350° C. for 12 hours. The BET surface area was determined from the N₂ adsorption isotherm in the pressure range of p/p₀=0.01-0.1 (S. Brunauer, P. H. Emmett, E. Teller, J. Am. Chem. Soc. 60 (1938) 308.) The t-plot method was used for the determination of micropore volume (B. C. Lippens, J. H. de Boer, J. Catal. 4 (1965), 309). The mesopore distribution was determined from the desorption isotherm using the Barret-Joyner-Hallenda pore size model (E. P. Barriet, L. G. Joyner, P. H. Hallenda, J. Am Chem. Soc. 78 (1951) 373).

### Acidity

Using a TA instruments Q500 TGA, samples were activated at 500° C. and then cooled to 200° C. and exposed to n-propylamine. The temperature was ramped to 280° C. to remove physisorbed amine, and then increased to 500° C. (at 10° C./minute). Brønsted acidity was determined by the amount of amine desorbed between 280° and 500° C.

### Materials

A commercially available ZSM-5 extrudate catalyst CBV 2314 from Zeolyst International was used as comparative example 4. The catalyst contained a zeolite with a silica to alumina mole ratio (SAR) of 23 and a binder. Binder-free ZSM-5 used in examples 1-3 and 5-6 is described below.

### Binder-Free ZSM-5 Synthesis

### Examples 1-3, 5-6

100% zeolite extrudates were prepared according to general procedures described in U.S. Pat. No. 5,558,851, U.S. Pat. No. 6,261,545 and U.S. Pat. No. 6,632,415 in which the reaction mixture contains only sufficient water to allow extrusion. Silica (Hi-Sil® 233 or Cab-O-Sil®) was combined with sodium aluminate, sodium hydroxide (5 N), water, and optionally tetrapropylammonium bromide and seed (for example, ZSM-5 Zeolyst CBV 3024E from Zeolyst International, added at 3% w relative to the dry ingredients) in a heated double armed mixer. The range of molar compositions of the mixture used in these preparations is given below.
SiO₂/Al₂O₃=23-38
TPA+/SiO₂=0-0.02
OH—/SiO₂=0.095-0.225
Na+/SiO₂=0.095-0.225
H₂O/SiO₂=4.8-8.2

Heating and mixing were continued until the mixture was dried to an extrudable state. The LOI of the mixture (volatiles measured at 550° C.) prior to extrusion ranged from 44-60%. The mixture was then extruded through 1/16″ die using a Bonnot extruder. In some cases, the extrudates were further dried at 75° C. in a ½″ layer in a screened tray to reduce the LOI to 37-60%. The extrudates were then loaded into an Abbe Rota-Cone vessel rated at 200 psig and 190° C. as described in U.S. Pat. No. 6,632,415. After sealing the vessel, rotation was started (0.5 rpm) and the jacket oil temperature was increased to give an internal temperature of 145-170° C. The reactor pressure ranged from 65-80 psig. After 40-72 hours, the vessel was cooled and vented. The crystallized extrudates were removed and washed at least three times with water (5 g water/g dry extrudate, 60-70° C.). The extrudates were then dried on a ½″ layer on a screen overnight at 120° C. and calcined by ramping from 125° C. to 550° C. at 1° C./min and holding at 550° C. for 4 hours. The extrudates were then ion-exchanged using a circulating 1-3 M ammonium nitrate solution for 2 hours at 60° C. The ion exchange was repeated up to 3 times followed by a final water wash. The ammonium form of the extrudate was then dried and calcined as described above. XRD of the resulting extrudate showed high crystallinity ZSM-5 with no impurities. ZD15033, ZD15034, and CBV 2314 are available from Zeolyst International.

TABLE 2

| | | | | | | | Mesopore | | |
|---|---|---|---|---|---|---|---|---|---|
| | | | | | | t-plot | Volume, | | |
| | | | | | BET | micropore | cc/g | | Brønsted |
| | Catalyst | Catalyst | Na₂O | SiO₂/Al₂O | S.A. | volume, | 50-1000 Å | CS | acidity |
| Example | Type | Name | % wt. | 3 mol/mol | m²/g | cc/g | width | lb/mm | mmole/g |
| 1 | Binder-Free | ZD15033-1 | 0.09 | 20.6 | 399 | 0.114 | 0.166 | 1.9 | 0.94 |
| | ZSM-5 | | | | | | | | |
| | extrudate | | | | | | | | |
| 2 | Binder-Free | ZD15033-2 | 0.16 | 24.8 | 382 | 0.085 | 0.106 | 2.9 | 0.91 |
| | ZSM-5 | | | | | | | | |
| | extrudate | | | | | | | | |
| 3 | Binder-Free | ZD15033-3 | <0.04 | 25.0 | 406 | 0.098 | 0.074 | 1.1 | 0.93 |
| | ZSM-5 | | | | | | | | |
| | extrudate | | | | | | | | |
| 4 | CSV 2314 | CBV 2314 | | 23* | 372 | 0.085 | 0.288 | | 0.66 |
| | (Extrudate | | | | | | | | |
| | with Binder) | | | | | | | | |
| 5 | Binder-Free | ZD15034-1 | 0.04 | 28.5 | 407 | 0.085 | 0.156 | 1.8 | 0.76 |
| | ZSM-5 | | | | | | | | |
| | extrudate | | | | | | | | |
| 6 | Binder-Free | ZD15034-2 | 0.05 | 34.0 | 410 | 0.102 | 0.076 | 0.9 | 0.65 |
| | ZSM-5 | | | | | | | | |
| | extrudate | | | | | | | | |

*result for CBV 2314 zeolite powder (not extrudate).
Na, Si and Al were analyzed by ICP analysis of the completely digested sample.

### Example A-F: Condensation

A model feed was prepared from water, 2-propanol, THF, acetone, acetic acid, 1,3 propanediol in a weight ratio of 70:15:7:4:3:1. Feed was flowed continuously over catalyst bed with catalysts from Table 2 as described below with frequent regenerations via coke burn. All the reactions were carried out at a temperature of 375° C. with a nitrogen pressure of 100 psig and a flow rate of 1 WHSV based on the total feed in a ½ inch×10-inch reactor bed operated downflow. The reaction was sampled twice daily with coke burns 1-2 times/week. The reaction was stopped when the catalyst stopped producing significant fuel (organic phase of the product exiting the reaction bed) or the fuel was of lower quality (low aromatics, high olefin content, etc.) The product contained an organic phase and an aqueous phase (total product was the combination of both organic phase and aqueous phase).

Catalyst for examples A-F were provided as follows:
Comparative Catalyst for Run A—Example 4
Catalyst for Run B—Example 1
Catalyst for Run C—Example 6
Catalyst for Run D—Example 2
Catalyst for Run E—Example 5
Catalyst for Run F—Example 3

The average fuel yield for each run was calculated for each run as: % weight of organic phase/% weight of total product

TABLE 3

| Run | A | B | C | D | E | F |
|---|---|---|---|---|---|---|
| Ave. organics | 14.38 | 13.27 | 5.93 | 11.9 | 9.07 | 13.6 |
| yield, % wt. | | | | | | |

As can been seen from the results Run B, Run D, and Run F provide similar average organics yield compared to conventional comparative catalyst run A. By contrast Run C using catalyst example 6 having a SAR of 34.0 yielded significantly less organics.

The concentration of 2-methylbutene was measured by Gas Chromatography and the concentration (by GC area %) over time (sample #) was plotted in FIG. 2 for runs B, D and F and Comparative Example A. 2-methylbutene was used as a flag molecule for loss of active sites. The concentration of 2-methylbutene increases as coke is deposited on the catalyst. After regeneration, it should return to baseline concentration. The chart shows the continued build of 2-methylbutene over time as the regenerations become progressively less effective in restoring the catalyst to the original activity of the fresh catalyst. As can been seen, the catalyst run D shows significant increase in 2-methylbutene that equates to permanent loss in aromatics production activity. By comparison, run B and run F of the invention show significantly slower 2-methylbutene concentration increase indicating the retention of activity for these novel catalysts.

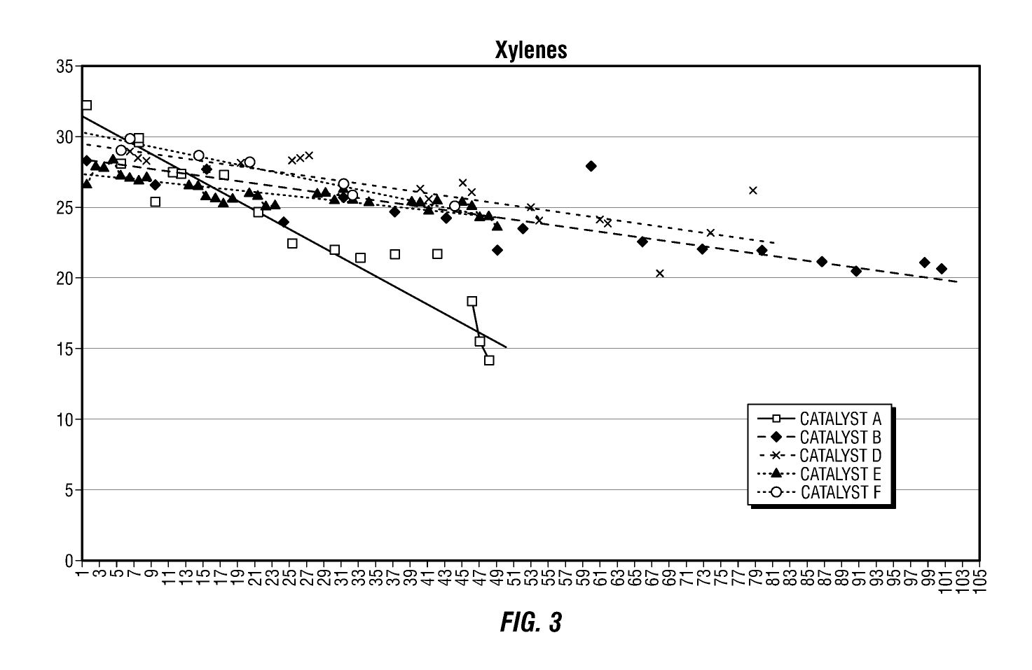

The concentration of xylenes was measured by Gas Chromatography and the concentration (by GC area %) over time (sample #) was plotted in FIG. 3 for runs B, D, E and F and Comparative Example A. The organic phase (fuel) produced by the catalyst can vary in quality. By tracking xylenes as a marker for aromatics formation, fuel quality is measured. As the catalyst activity is lost over time, the organic phase fuel becomes progressively less aromatic and more olefinic. This loss in fuel quality would not be seen by tracking only organic phase fuel quantity. As can been seen, the comparative catalyst run A using conventional catalyst shows significant loss of xylene concentration after about 45 to 47 samples (approximately 30 days) whereas runs B, D, E and F using the binder-free catalyst of the invention process shows much slower decrease in xylene concentration indicating retention of catalytic activity (i.e., increased or longer catalyst life).

As can be seen from the data above, run B using catalyst example 1 and run F using catalyst example 3 particularly exhibit much longer catalyst life compared to conventional catalyst run A, and further retain excellent aromatics production activity.

We claim:

1. A process for the production of a higher hydrocarbon from solid biomass, said process comprising:
a. providing a biomass solid containing cellulose, hemicellulose, and lignin;
b. digesting and hydrodeoxygenating the biomass solid in a liquid digestive solvent in the presence of a hydrothermal hydrocatalytic catalyst and hydrogen at a temperature in the range of 110° C. to less than 300° C. and at a pressure in a range of from 20 bar to 200 bar, to form a stable oxygenated hydrocarbon intermediate product, said stable oxygenated hydrocarbon intermediate product having at least 60% of carbon in molecules having 9 carbon atoms or less; and
c. contacting at least a portion of the stable oxygenated hydrocarbon intermediate product with a binder-free shaped ZSM-5 catalyst having zeolite content of greater than 98%, silica to alumina molar ratio of at most 28 to 1, and at a temperature in the range from 325° C. to about 425° C. producing water and an aromatics-rich higher hydrocarbons stream having at least 50 wt. % of aromatics containing hydrocarbon based on the aromatics-rich hydrocarbons stream.

2. The method of claim 1 wherein reacting at least a portion of the stable oxygenated hydrocarbon intermediate product with an acidic amorphous silica alumina catalyst at a temperature in the range from 300° C. to 400° C. prior to step c.

3. The method of claim 1 wherein the binder-free shaped ZSM-5 catalyst is a binder-free ZSM-5 extrudate.

4. The method of claim 1 wherein the binder-free shaped ZSM-5 catalyst has a BET surface area in the range of 300 m²/g to 500 m²/g, preferably in the range of 350 m²/g to 450 m²/g.

5. The method of claim 1 wherein the binder-free shaped ZSM-5 catalyst has a Na₂O content of less than 0.15% wt.

6. The method of claim 1 wherein the binder-free shaped ZSM-5 catalyst has a crush strength of 0.5 lb/mm or greater, preferably 1 lb/mm or greater.

7. The method of claim 1 wherein the binder-free shaped ZSM-5 catalyst has Brønsted acidity of at least 0.80 mmole/g.

8. The method of claim 1 wherein the binder-free shaped ZSM-5 catalyst has a mesopore volume in the pore width range of 50 Å to 1000 Å of at least 0.07 cc/g.

9. The method of claim 1 wherein the solid biomass is a lignocellulosic biomass.

10. The method of claim 1 further comprising (d) regenerating the binder-free shaped ZSM-5 catalyst from step (c) at a temperature of about equal or greater than 390° C. in the presence of an oxygen-containing gas thereby producing a regenerated catalyst and further conducting step (c) using such regenerated catalyst.

11. The method of claim 1 wherein the binder-free shaped ZSM-5 catalyst has a silica to alumina molar ratio of at most 27 to 1, more preferably at most 26:1, even more preferably at most 25:1.

12. The method of claim 7 wherein the binder-free shaped ZSM-5 catalyst has a Brønsted acidity of at least 0.85 mmole/g.

13. The method of claim 5 wherein the binder-free shaped ZSM-5 catalyst has a Na₂O content of at most 0.10% wt.

14. The method of claim 10 wherein said regeneration is carried out at least 10 times.

15. A process for the production of a higher hydrocarbon from solid biomass, said process comprising:
a. providing a biomass solid containing cellulose, hemicellulose, and lignin;
b. digesting and hydrodeoxygenating the biomass solid in a liquid digestive solvent in the presence of a hydrothermal hydrocatalytic catalyst and hydrogen at a temperature in the range of 110° C. to less than 300° C. and at a pressure in a range of from 20 bar to 200 bar, to form a stable oxygenated hydrocarbon intermediate product, said stable oxygenated hydrocarbon intermediate product having at least 60% of carbon in molecules having 9 carbon atoms or less;
c. contacting at least a portion of the stable oxygenated hydrocarbon intermediate product with a binder-free shaped ZSM-5 catalyst having zeolite content of greater than 98%, silica to alumina molar ratio of at most 28 to 1 and an Brønsted acidity of at least 0.80 mmole/g., at a temperature in the range from 325° C. to about 425° C. producing water and an aromatics-rich higher hydrocarbons stream having at least 50 wt % of aromatics containing hydrocarbon based on the aromatics-rich hydrocarbons stream; and
d. regenerating the binder-free shaped ZSM-5 catalyst from step (c) at a temperature of about equal or greater than 390° C. in the presence of an oxygen-containing gas thereby producing a regenerated catalyst and further conducting step (c) using such regenerated catalyst.
